(19) United States
(12) Patent Application Publication
(10) Pub. No.: US 2012/0136371 A1
(43) Pub. Date: May 31, 2012

(54) SYSTEMS AND METHODS FOR MEDICAL TELEPRESENCE

(76) Inventor: Ivar Mendez, Lucasville (CA)

(21) Appl. No.: 13/302,800

(22) Filed: Nov. 22, 2011

Related U.S. Application Data
(60) Provisional application No. 61/417,351, filed on Nov. 26, 2010.

(51) Int. Cl. A61B 19/00; H04N 7/14

(52) U.S. Cl. 606/130; 348/14.04

(57) ABSTRACT

A medical telepresence system includes, at a first location, a local video camera having a field of view and a medical treatment device having a device display for displaying real-time information and also having a device control interface. The local video camera is coupled to a communications link for transmitting video images to a second location that is physically remote from the first location. The medical treatment device is positioned so that the device display and the device control interface are within the field of view of the local video camera and the field of view also simultaneously accommodates a patient. The local video camera can be a robot video camera carried by a mobile telepresence robot, which may include a physical manipulator or a manually positionable arm to hold the medical treatment device.

### CROSS-REFERENCE TO RELATED APPLICATION

This application claims priority to U.S. Provisional Patent Application No. 61/417,351 filed on Nov. 26, 2010.

### TECHNICAL FIELD

This disclosure describes telepresence systems and methods, and more particularly medical telepresence systems and methods.

### BACKGROUND

Broadly speaking, telepresence systems are systems which enable a person who is physically present in one location to mimic some of the aspects of being present at another physically remote location. Telepresence systems may or may not include interactive features. For example, a television broadcast of a live event, such as a baseball or soccer game, is a simple non-interactive form of telepresence since the viewer can see the game but cannot interact with it, while a telephone network is a simple interactive telepresence system since each user can interact with the other user. Interactive telepresence systems have promise in the field of medicine, since they could enable a medical specialist to treat patients in a variety of physical locations, without risking the patient's health, or wasting the specialist's valuable time, with the delays associated with travel between locations. However, in order to achieve this promise, such telepresence systems must be able to achieve the necessary physical interaction between doctor and patient while also retaining the personal doctor-patient relationship.

### SUMMARY

A method for enabling a medical practitioner to be telepresent at a first location when the medical practitioner is physically present at a second location that is physically remote from the first location comprises providing, at the second location, a remote station including a remote station video display and providing a local video camera at the first location. The local video camera has a field of view and is coupled to the remote station video display to transmit video images thereto. The method further comprises causing a patient at the first location to be within the field of view of the local video camera, and providing a medical treatment device at the first location. The medical treatment device has a device display for displaying real-time information and a device control interface. The method comprises causing the medical treatment device to be positioned, relative to the local video camera, so that the device display and the device control interface are within the field of view of the local video camera simultaneously with the patient being within the field of view of the local video camera.

In one embodiment, the step of providing a local video camera at the first location comprises providing a mobile robot at the first location, in which embodiment the local video camera is a robot video camera carried by the robot. In this embodiment, the step of causing a patient to be within the field of view of the local video camera comprises positioning the mobile robot so that the patient is within the field of view of the robot video camera, and the step of causing the medical treatment device to be positioned, relative to the local video camera, so that the device display and the device control interface are within the field of view of the local video camera, comprises positioning the medical treatment device relative to the robot video camera. The method may further comprise activating a two-way audio communication channel between the medical practitioner and a human assistant at the first location so that the human assistant can manipulate the device control interface in accordance with instructions from the medical practitioner.

In a particular implementation, the robot includes a first physical manipulator, the remote station includes a remote station control interface coupled to the first physical manipulator to remotely control the first physical manipulator, and the device control interface is positioned within a range of motion of the first physical manipulator so that the first physical manipulator can manipulate the device control interface. In one particular instance of this implementation, the robot includes a second physical manipulator, the remote station control interface is coupled to the second physical manipulator to remotely control the second physical manipulator, and the medical treatment device is grasped by the second physical manipulator. In another particular instance of this implementation, a manually positionable arm is secured to the robot and the step of positioning the medical treatment device relative to the robot video camera comprises positioning the manually positionable arm on the robot so that a receptacle on the arm is within the field of view of the robot video camera, and securing the medical treatment device in the receptacle. These steps may be carried out in any order.

The method may further comprise providing a local video display at the first location, with the remote station including a remote station video camera coupled to the local video display to transmit video images thereto. The local video camera and the local video display are arranged relative to one another so that causing the patient at the first location to be within the field of view of the local video camera also causes the local video display to face the patient. In a particular embodiment, in which a mobile telepresence robot is used, the local display is a robot video display carried by the robot.

A medical telepresence system comprises a local video camera at a first location and having a field of view. The local video camera is coupled to a communications link for transmitting video images to a second location that is physically remote from the first location. A medical treatment device having a device display for displaying real-time information and a device control interface is also at the first location. The medical treatment device is positioned so that the device display and the device control interface are within the field of view of the local video camera and the field of view also simultaneously accommodates a patient. The medical telepresence system may further comprise a two-way audio communication channel between the first location and the second location.

In a particular embodiment of the medical telepresence system, the local video camera is a robot video camera carried by a mobile telepresence robot. The robot includes a robot control system coupled to the communications link, the robot video camera is coupled to the robot control system and thereby coupled to the communications link, and the robot includes a locomotion system coupled to the robot control system. The medical telepresence system further comprises a remote station at the second location and which is coupled to the communications link and thereby coupled to the robot control system to control the robot.

In one implementation, the robot includes a first physical manipulator coupled to the robot control system, the remote station includes a remote station control interface coupled to the first physical manipulator through the communications link and the robot control system to remotely control the first physical manipulator, and the device control interface is positioned within a range of motion of the first physical manipulator so that the first physical manipulator can manipulate the device control interface. In a more particular implementation, the robot includes a second physical manipulator coupled to the robot control system, the remote station control interface is coupled to the second physical manipulator through the communications link and the robot control system to remotely control the second physical manipulator, and the medical treatment device is grasped by the second physical manipulator.

In another implementation, a manually positionable arm is secured to a body of the robot, the arm has a receptacle for securely receiving the medical treatment device, the medical treatment device is securely received in the receptacle, and the arm is positioned so that the receptacle is within the field of view of the robot video camera.

The medical telepresence system may further comprise a local video display at the first location, with the remote station including a remote station video camera coupled to the local video display to transmit video images thereto, with the local video camera and the local video display being arranged relative to one another so that causing a patient at the first location to be within the field of view of the local video camera also causes the local video display to face the patient. In a particular embodiment, in which a mobile telepresence robot is used, the local display is a robot video display carried by the robot.

A mobile medical telepresence robot comprises a robot video camera having a field of view, with the robot video camera being coupled to a communication unit for transmitting video images to a physically remote location. A manually positionable arm is secured to the body of the robot, with the arm having a receptacle for securely receiving a medical treatment device. At least one of the arm and the robot video camera is positionable so that the receptacle is within the field of view of the robot video camera while a patient is also within the field of view of the robot video camera. A medical treatment device having a device display for displaying real-time information and a device control interface can be securely received in the receptacle and the arm and the robot video camera can be positioned relative to one another so that the device display and the device control interface are within the field of view of the robot video camera. The mobile medical telepresence robot will typically comprise the robot body, the robot video camera, the communication unit, a robot control system coupled to the communication unit and a locomotion system coupled to the robot control system. The robot video camera is coupled to the robot control system and thereby coupled to the communication unit, which is operable to receive and transmit external communications. The mobile medical telepresence robot may further comprise a robot video display coupled to the robot control system and thereby coupled to the communication unit to display images from a remote video camera.

### BRIEF DESCRIPTION OF THE DRAWINGS

These and other features will become more apparent from the following description in which reference is made to the appended drawings wherein:

FIG. 1 is a schematic representation of a first exemplary medical telepresence system;

FIG. 2 is a schematic representation of a second exemplary medical telepresence system;

FIG. 3 is a schematic representation of a third exemplary medical telepresence system;

FIG. 4 is a schematic representation of a fourth exemplary medical telepresence system;

FIG. 5 is a schematic representation of a fifth exemplary medical telepresence system;

FIG. 6 is a schematic representation of a sixth exemplary medical telepresence system;

FIG. 7 shows an exemplary physical embodiment of a mobile medical telepresence robot having an exemplary manually positionable arm and which may be used as part of the exemplary medical telepresence system of FIG. 6;

FIG. 8 is a perspective view of the manually positionable arm of FIG. 7;

FIG. 9A is an exploded perspective view of the manually positionable arm of FIG. 7;

FIG. 9B shows attachment of the manually positionable arm of FIG. 7 to the mobile medical telepresence robot of FIG. 7;

FIG. 10 is a side view of the manually positionable arm of FIG. 7;

FIG. 11 is a top plan view of the manually positionable arm of FIG. 7;

FIG. 12 is a side view the mobile medical telepresence robot of FIG. 7 and the manually positionable arm of FIG. 7, showing various positions of the manually positionable arm;

FIG. 13 shows the mobile medical telepresence robot of FIG. 7 with the manually positionable arm of FIG. 7 holding an exemplary medical treatment device in use with a patient;

FIG. 14 is a flow chart showing a method for enabling a medical practitioner to be telepresent at a first location when the medical practitioner is physically present at a second location that is physically remote from the first location; and

FIG. 15 is a schematic representation of an exemplary computer system.

### DETAILED DESCRIPTION

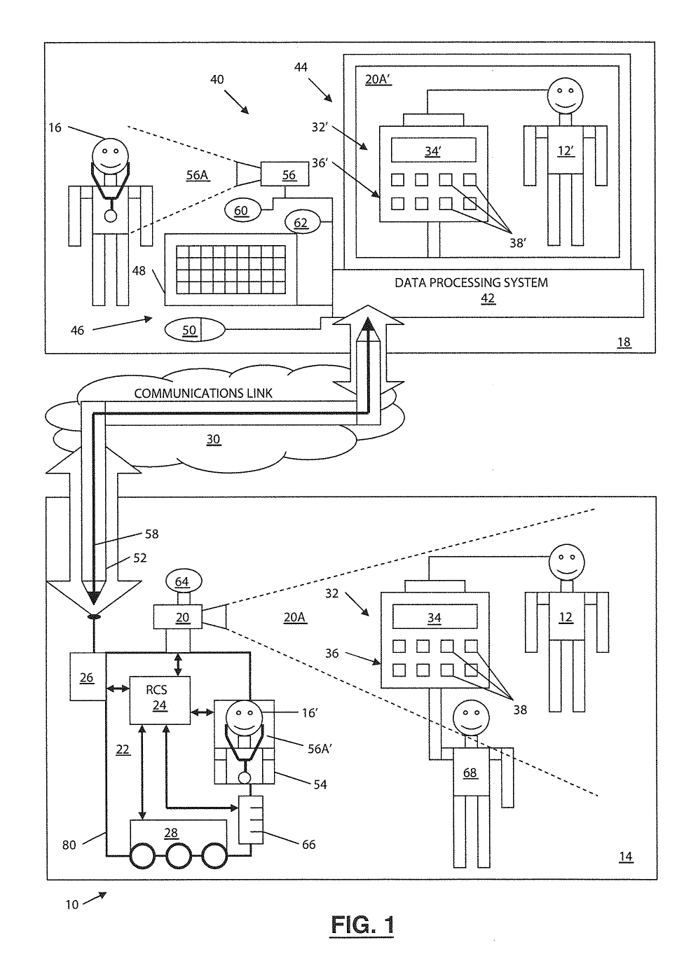

Reference is now made to FIG. 1, which is a schematic representation of a first exemplary medical telepresence system, denoted generally by the reference numeral 10. The telepresence system 10 enables a human patient 12 present at a first location 14 to receive certain types of treatment from a medical professional 16, such as a doctor, who is physically present at a second location 18 that is physically remote from the first location 14. For example, the first location may be a first hospital and the second location may be a second hospital that is tens, hundreds or even thousands of kilometers from the first hospital.

The medical telepresence system 10 comprises a local video camera 20 positioned in the first location 14 and having a field of view 20A. In the illustrated embodiment, the local video camera 20 is a robot video camera 20 carried by a mobile telepresence robot 22 although the local video camera 20 may also be a stand-alone camera. As used herein, the term “robot” is intended to encompass autonomous and semiautonomous systems, as well as non-autonomous, teleoperated (remote controlled) systems. The robot 22, and hence the robot video camera 20 that it carries, are physically present at the first location 14. The robot 22 includes, in addition to the robot video camera 20, a robot control system (RCS) 24 for controlling various subsystems of the robot 22, a communication unit 26 and a locomotion system 28 enabling the robot 22 to travel about the first location 14, all supported by a robot body 80. The communication unit 26 is coupled to a communications link 30, such as the Internet or a private network, and may be, for example, a wireless transmitter, such as a wireless Ethernet connector, or another suitable connection. The robot control system 24 is coupled to the communication unit 26 and is thereby coupled to the communications link 30 and the robot video camera 20 is coupled to the robot control system 24 and thereby coupled to the communications link 30, via the communication unit 26, so that the robot video camera 20 can transmit video images to the second location 18. The robot control system 24 is also coupled to the locomotion system 28 to control movement of the robot 22.

A medical treatment device 32 is also present at the first location 14. In the particular illustrative embodiment of a medical telepresence system 10 shown in FIG. 1, the medical treatment device 32 is a programmer unit for a neurostimulator device (not shown) implanted in the patient 12; other types of medical treatment devices may also be used. The a medical treatment device 32 has a device display 34 for displaying real-time information relating to the treatment and a device control interface 36, in the form of buttons 38, for controlling the medical treatment device 32 and hence the treatment administered thereby. As can be seen in FIG. 1, the medical treatment device 32 is positioned so that both the device display 34 and the device control interface 36 are within the field of view 20A of the robot video camera 20 and the field of view 20A also simultaneously accommodates the patient 12.

The medical telepresence system 10 further comprises a remote station 40 at the second location 18. The remote station 40 is coupled to the communications link 30 and thereby coupled to the robot control system 24 so that it can transmit communications to the robot control system 24, and receive communications from the robot control system 24. For example, in the illustrated embodiment the remote station 40 comprises a data processing system 42, which may be a suitably programmed general purpose computer, and where the communications link 30 is the Internet or a private network, may be coupled thereto by way of an Ethernet connection or other suitable connection. The data processing system 42 of the remote station 40 may include a communication unit (not specifically illustrated in FIG. 1), such as an Ethernet card, to facilitate communications via the communications link 30.

In the particular illustrative embodiment of a medical telepresence system 10 shown in FIG. 1, the data processing system 42 has a remote station display 44 and a remote station control interface 46 in the form of keyboard 48 and pointing device 50. Other physical hardware may also be used for the remote station control interface 46, including, without limitation, a touch screen display and/or a joystick. For example, a joystick could be used to generate control signals that can be transmitted from the remote station 40 through the communications link 30 to the robot control system 24, which can then use those control signals to generate instructions to the locomotion system 28 of the robot 22 so as to navigate the robot 22 about the first location 14.

The robot control system 24 transmits a video signal 52 to the remote station 40, and more particularly the data processing system 42, via the communication unit 26 and the communications link 30, so that the robot video camera 20 can transmit video images to the second location 18. The video images transmitted to the remote station 40 are then displayed on the remote station display 44 as an image 20A′ corresponding to the field of view 20A of the robot video camera 20. As can be seen in FIG. 1, the image 20A′ shown on the remote station display 44 includes an image 32′ of the medical treatment device 32, including image portions 34′, 36′ and 38′ corresponding, respectively, to the device display 34 and device control interface 36 in the form of buttons 38, as well as an image 12′ of the patient 12.

Preferably, a medical telepresence system includes a local video display at the first location for displaying video images from the second location. In the illustrated embodiment shown in FIG. 1, the medical telepresence system 10 includes a local video display in the form of robot video display 54 forming part of the robot 22 and coupled to the robot control system 24, and the remote station 40 includes a remote station video camera 56 having a field of view 56A. Alternatively, a stand-alone local video display may be used. The remote station video camera 56 is coupled to the data processing system 42, and is thereby coupled, via the communications link 30, communication unit 26 and robot control system 24, to the robot video display 54 so that the remote station video camera 56 can transmit video images to the robot video display 54. As shown in FIG. 1, the field of view 56A of the remote system video camera 56 includes the medical professional 16, who is positioned in front of and is using the remote station 40, and consequently the robot video display 54 includes an image 56A′ corresponding to the field of view 56A of the remote station video camera 56, which image 56A′ includes an image 16′ of the medical professional 16. As illustrated schematically in FIG. 1, the local video camera, in particular the robot video camera 20, and the local video display, in particular the robot video display 54, are arranged relative to one another so that causing the patient 12 at the first location 14 to be within the field of view of the local video camera, namely the robot video camera 20, also causes the local video display, namely the robot video display 54 to face the patient 12. More particularly, the robot video camera 20 and the robot video display 54 are arranged relative to one another so that positioning the robot 22 so that the patient 12 is within the field of view 20A of the robot video camera 20 also positions the robot video display 54 to face the patient 12.

Preferably, as shown schematically in FIG. 1, the medical telepresence system 10 includes a two-way audio communication channel 58 which enables audio communication between the first location 14 and the second location 18. The two-way audio communication channel 58 may form part of the video signal 52, as shown, or may comprise a separate signal. For example, existing telephone communication networks may be used to provide a two-way audio communication channel. In the embodiment shown in FIG. 1, the remote station 40 includes a remote station microphone 60 and remote station speaker 62 each coupled to the data processing system 42 of the remote station 40, and the robot 22 includes a local microphone in the form of robot microphone 64 and a local speaker in the form of robot speaker 66, each of which is coupled to the robot control system 24. Alternatively, a stand-alone local speaker and local microphone may be used. Accordingly, audio signals from the remote station microphone 60 can be transmitted, via the data processing system 42, communications link 30, communication unit 26 and robot control system 24, to the robot speaker 66. Conversely, audio signals from the robot microphone 64 can be transmitted, via the robot control system 24, communication unit 26, communications link 30 and data processing system 42, to the remote station speaker 62. Thus, audio communications can be transmitted between the first location 14 and the second location 18, thereby enabling the patient 12 and the medical professional 16 to talk to one another. The robot microphone 64 is shown as forming part of the robot video camera 20, in other embodiments the robot microphone may be a separate component.

As noted above, the medical treatment device 32 is positioned so that its device display 34 and device control interface 36 are within the field of view 20A of the robot video camera 20 while the patient 12 is also simultaneously within the field of view 20A. In the particular illustrated embodiment shown in FIG. 1, this positioning of the medical treatment device 12 is accomplished by having a medical staff member 68 hold the medical treatment device 12 within the field of view 20A of the robot video camera 68. The medical staff member 68 may be, for example, a nurse or an orderly, or may be a general practice physician who is working with the medical professional 16, who would typically be a specialist. The use of the terms “medical professional” and “medical staff member” are not intended to imply superiority of a specialist over a general practitioner, but merely to express the distinction between the individuals at the first location and at the second location, since the individual at the second location will typically, although not necessarily, be providing a specific medical expertise not possessed by the individual present at the first location. For example, the medical professional 16 may be a neurologist or a neurosurgeon, and the medical staff member 68 could be either a non-specialist such as a family practice physician, or a specialist in different field, such as an orthopedic surgeon who is consulting with the neurologist or neurosurgeon on specific treatments requiring both sets of expertise.

Advantageously, because the device display 34, device control interface 36 and patient 12 are all within the field of view 20A of the robot video camera 20, the image 20A′ shown on the remote station display 44 includes image portions 34′, 36′ and 38′ corresponding, respectively, to the device display 34 and device control interface 36 in the form of buttons 38, as well as an image 12′ of the patient 12. As a result, the medical professional 16 can remotely “see” the device display 34, device control interface 36 and patient 12 simultaneously. This enables the medical professional 16 to provide instructions, for example by way of the two-way audio communication channel 58, to the medical staff member 68 as to how to manipulate the device control interface 36 (e.g. the buttons 38) and to observe, via the remote station display 44, the actual manipulation to confirm that the medical staff member 68 has manipulated the device control interface 36 correctly. As a potential further way to verify the correctness of the manipulation of the device control interface 36, the medical professional 16 can also observe, via the remote station display 44, the device display 34, which will typically include real-time information representative of the manipulation of the device control interface 36. Concurrently, the medical professional 16 can also observe, via the remote station display 44, the patient 12, and can therefore observe the reaction of the patient 12 to the treatment resulting from the manipulation of the device control interface 36. Thus, the medical telepresence system 10 allows the operator, e.g. the medical professional 16, to control programming of a medical treatment device, e.g. the medical treatment device 32, and observe a patient, e.g. patient 12, at the same time. Moreover, where the device display 34 includes real-time diagnostic information relating to the patient 12, such as heart rate, blood pressure or other information, the medical professional 16 can combine this information with her/his observations of the patient 12 to assist in diagnosis and/or further treatment. It is also contemplated that the patient 12 may be suitably positioned and may manipulate the device control interface 36 herself or himself, with the device display 34, device control interface 36 remaining within the field of view 20A of the robot video camera 20. Also, more than one medical treatment device 32 may be used simultaneously within the field of view 20A.

It should be noted here that the medical treatment device 32 is electrically remote from both the robot 22 and the remote station 40; that is, there is no direct communication between the medical treatment device 32 and either the robot 22 or the remote station 40. As a result, a medical telepresence system as described herein can be used with a variety of different types of medical treatment device. This obviates the need for complex software and/or hardware interfaces between the medical treatment device 32 and the robot 22 and/or the remote station 40, which would typically be specific to the particular type, and even to the particular manufacturer and model, of the medical treatment device 32.

In the particular exemplary embodiment shown in FIG. 1, the medical treatment device 32 is positioned so that the device display 34, device control interface 36 and patient 12 are all within the field of view 20A of the robot video camera 20 by having the medical staff member 68, or the patient 12, hold the medical treatment device in an appropriate position. In other embodiments, other techniques may be used to position a medical treatment device relative to a patient, some examples of which are described in greater detail below.

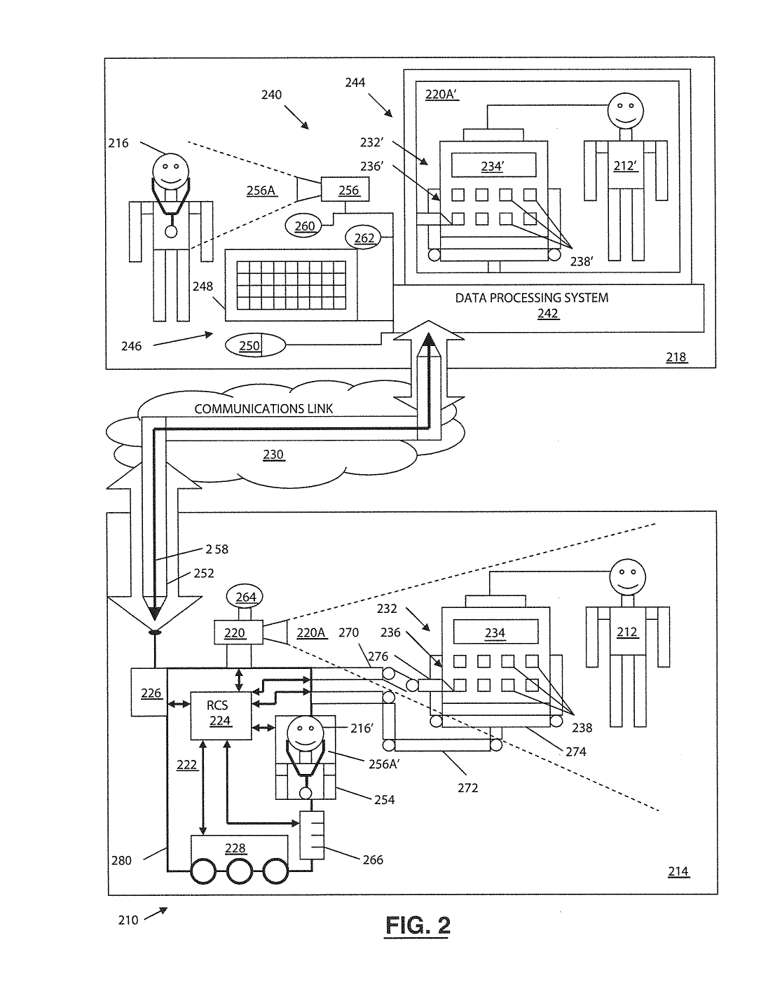

Reference is now made to FIG. 2, in which a second exemplary embodiment of a medical treatment device is shown generally at 210. The second embodiment 210 is identical to the first embodiment 10, except that instead of having the medical staff member or the patient hold the medical treatment device in an appropriate position, the robot includes at least one manipulator, such as a powered and articulated arm with a gripper such as a claw, which holds the medical treatment device. Accordingly, corresponding reference numerals are used to refer to features of the medical telepresence system 210 in FIG. 2 that correspond to features of the medical telepresence system 10 shown in FIG. 1, except with the prefix “2”. Thus, for example, in the medical telepresence system 210 shown in FIG. 2, the patient is denoted by reference numeral 212, the first location is denoted by reference numeral 214, the medical professional is denoted by reference numeral 216, the second location is denoted by reference numeral 218, the robot video camera (serving as a local video camera) is denoted by reference numeral 220, the robot is denoted by reference numeral 222, and so on.

As can be seen in FIG. 2, the robot 222 includes first and second manipulators 270, 272 in the form of powered, articulated arms. The first and second manipulators 270, 272 are coupled to the robot control system 224, and the remote station control interface 246 is coupled to the first and second manipulators 270, 272 via the data processing system 242, the communications link 230, the communication unit 226 and the robot control system 224 so that the medical professional 216 can remotely control the first and second manipulators 270, 272 from the remote station 240. As can be seen in FIG. 2, the medical treatment device 232 is grasped by a gripper 274 forming part of the second physical manipulator 272 and held so that the device control interface 236 is positioned within the range of motion of the first physical manipulator 270 so that the first physical manipulator 270 can manipulate the device control interface 236, for example by pushing the buttons 238. To this end, the first physical manipulator may be provided with a specialized finger attachment 276. As shown in FIG. 2, the second physical manipulator 272 holds the medical treatment device 232 so that both the device display 234 and the device control interface 236 are within the field of view 220A of the robot video camera 220 with the field of view 220A also simultaneously accommodating the patient 212. Moreover, causing the patient 212 to be within the field of view 220A of the robot video camera 220 also causes the robot video display 254 to face the patient 212.

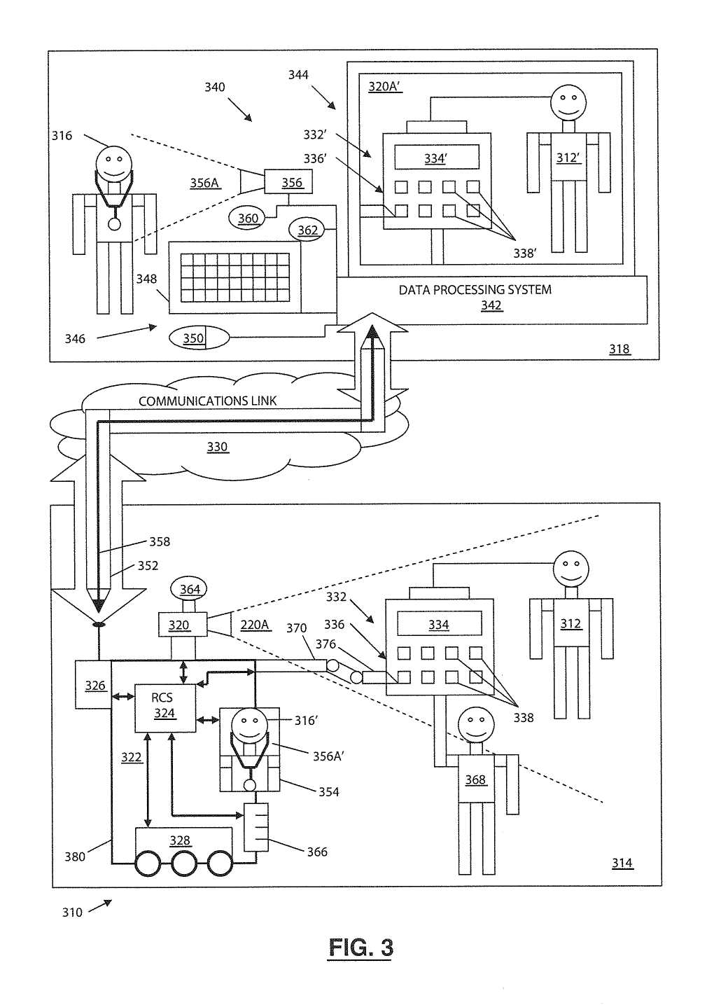

A third embodiment of a medical telepresence system is shown in FIG. 3, and is indicated generally by the reference numeral 310. The third embodiment 310 is identical to the second embodiment 210, except that the robot includes only a single manipulator. Accordingly, corresponding reference numerals are used to refer to features of the medical telepresence system 310 in FIG. 3 that correspond to features of the medical telepresence system 210 shown in FIG. 2, except with the prefix “3” instead of “2”.

In the third embodiment of a medical telepresence system 310, a medical staff member 368 holds the medical treatment device 332 in an appropriate position, so that both the device display 334 and the device control interface 336 are within the field of view 320A of the robot video camera 320 with the field of view 320A also simultaneously accommodating the patient 312. Alternatively, the patient 312 can hold the medical treatment device 332. The medical professional 316 can remotely control the single manipulator 370 from the remote station 340, and the medical staff member 368 holds the medical treatment device 332 so that the device control interface 336 is positioned within the range of motion of the single physical manipulator 370. The medical professional 316 can control the single physical manipulator 370 to manipulate the device control interface 336, for example by pushing the buttons 338 using the finger attachment 376. Again, causing the patient 312 to be within the field of view 320A of the robot video camera 320 also causes the robot video display 354 to face the patient 312.

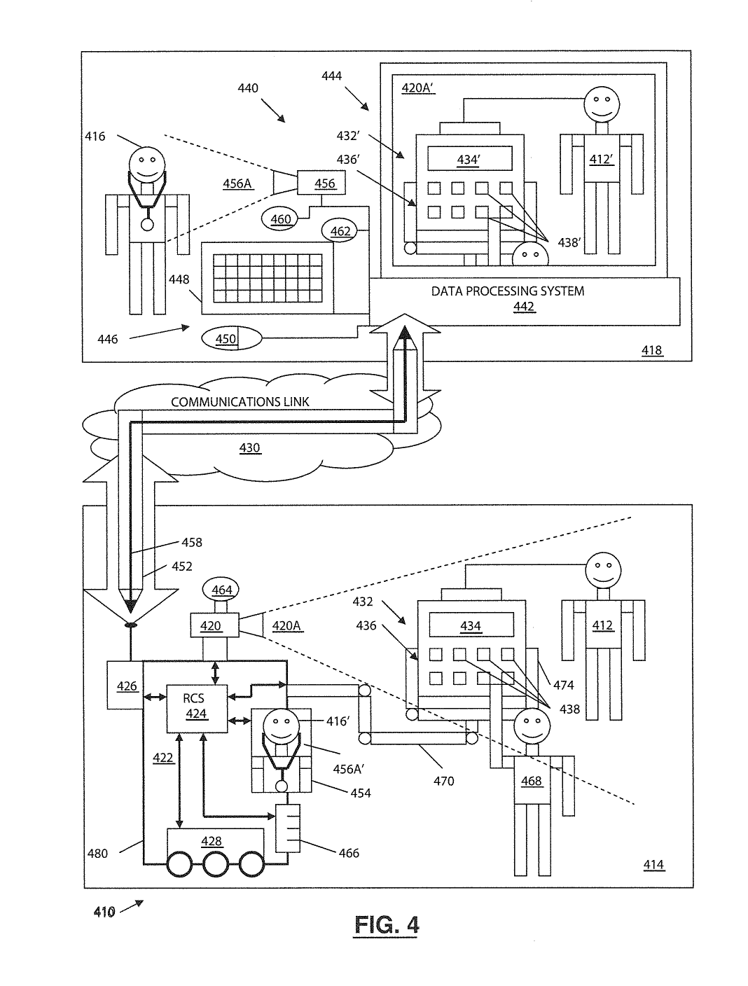

FIG. 4 shows a fourth embodiment 410 of a medical telepresence system. Corresponding reference numerals are used to refer to features of the medical telepresence system 410 in FIG. 4 that correspond to features of the medical telepresence system 310 shown in FIG. 3, except with the prefix “4” instead of “3”. The fourth embodiment 410 is identical to the third embodiment 310 except that instead of the specialized finger attachment 376, the single manipulator 470 includes a gripper 474 used to hold the medical treatment device 432 so that both the device display 434 and the device control interface 436 are within the field of view 420A of the robot video camera 420 with the field of view 420A also simultaneously accommodating the patient 412. The single manipulator 410 may alternatively be provided with a specialized receptacle for the medical treatment device 432 instead of the gripper 474. Causing the patient 412 to be within the field of view 420A of the robot video camera 420 also causes the robot video display 454 to face the patient 412. The medical staff member 468 manipulates the device control interface 436 in accordance with instructions from the medical professional 416.

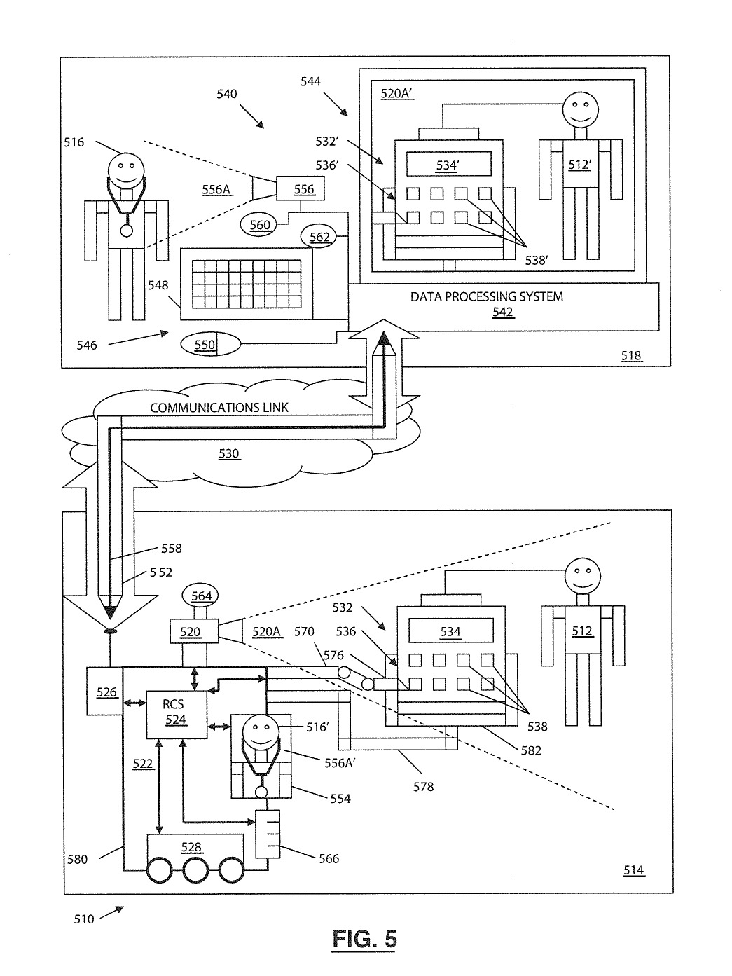

FIG. 5 shows a fifth embodiment of a medical telepresence system, which is indicated generally by the reference numeral 510. The fifth embodiment 510 is identical to the second embodiment 210, except that the robot includes only a single manipulator 570, with the second manipulator having been replaced by a manually positionable arm 578 secured to the body 580 of the robot 522. Accordingly, corresponding reference numerals are used to refer to features of the medical telepresence system 510 in FIG. 5 that correspond to features of the medical telepresence system 310 shown in FIG. 3, except with the prefix “5” instead of “3”.

The manually positionable arm 578 has a receptacle 582 for securely receiving the medical treatment device 532, and the medical treatment device 532 is securely received in the receptacle 582. The arm 582 is positioned so that the receptacle 582 is within the field of view 520A of the robot video camera 520, and oriented so that both the device display 534 and the device control interface 536 are within the field of view 520A of the robot video camera 520, with the field of view 520A also simultaneously accommodating the patient 512. As in the other embodiments, the robot video camera 520 and the robot video display 554 are positioned such that causing the patient 512 to be within the field of view 520A of the robot video camera 520 also causes the robot video display 554 to face the patient 512. The medical staff member 568, or the patient 512, can then manipulate the device control interface 536 (e.g. the buttons 538).

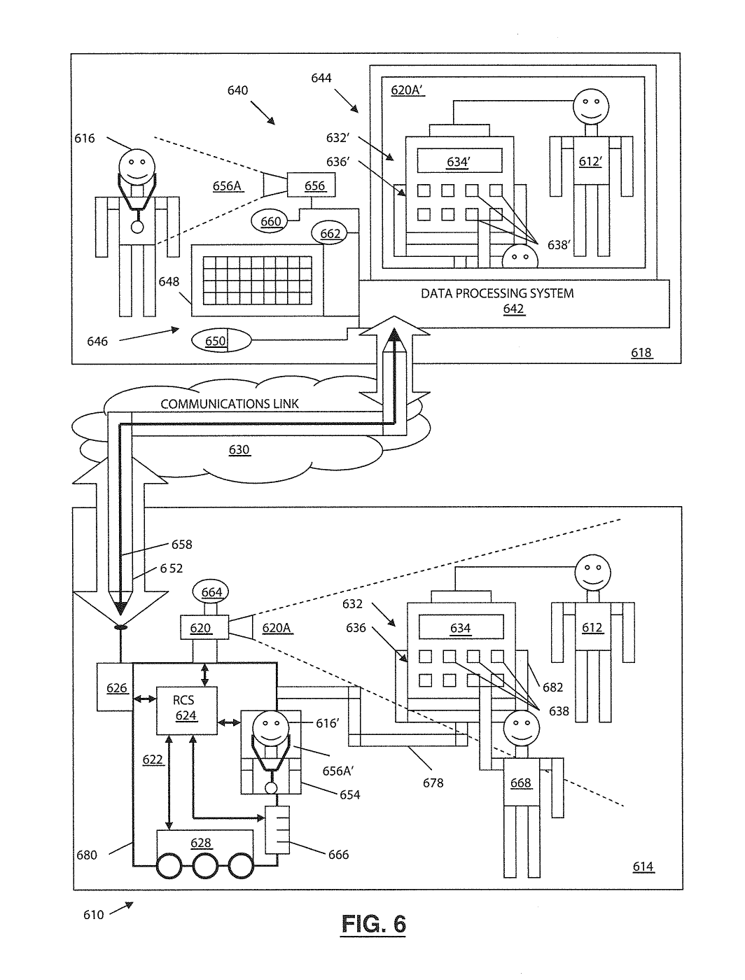

Now referring to FIG. 6, a sixth embodiment of a medical telepresence system is shown generally at 610. The sixth embodiment 610 is identical to the first embodiment 10, except that the robot is provided with a manually positionable arm identical to that shown and described in respect of the fourth embodiment 410 depicted in FIG. 4. Accordingly, corresponding reference numerals are used to refer to features of the medical telepresence system 610 in FIG. 6 that correspond to features of the medical telepresence systems 10 and 410 shown in FIGS. 1 and 4, respectively, except with the prefix “6” instead of “1” or “4”.

As in the fourth embodiment 410, in the sixth embodiment 610 the manually positionable arm 678 is secured to the robot body 680, and has a receptacle 682 for securely receiving the medical treatment device 632. The medical treatment device 632 is securely received in the receptacle 682, and the arm 678 is positioned so that the receptacle 682, and hence the medical treatment device 632, is within the field of view 620A of the robot video camera 620. The arm 678 and receptacle 682 are oriented so that both the device display 634 and the device control interface 636 are within the field of view 620A of the robot video camera 620 while the patient 612 is simultaneously accommodated within the field of view 620A. As in the first embodiment 10, in the sixth embodiment 610 the medical staff member 668 will manipulate the device control interface 636 (e.g. the buttons 638) according to instructions provided by the medical professional 616, for example by way of the two-way audio communication channel 658. As with the other embodiments, causing the patient 612 to be within the field of view 620A of the robot video camera 620 also causes the robot video display 654 to face the patient 612.

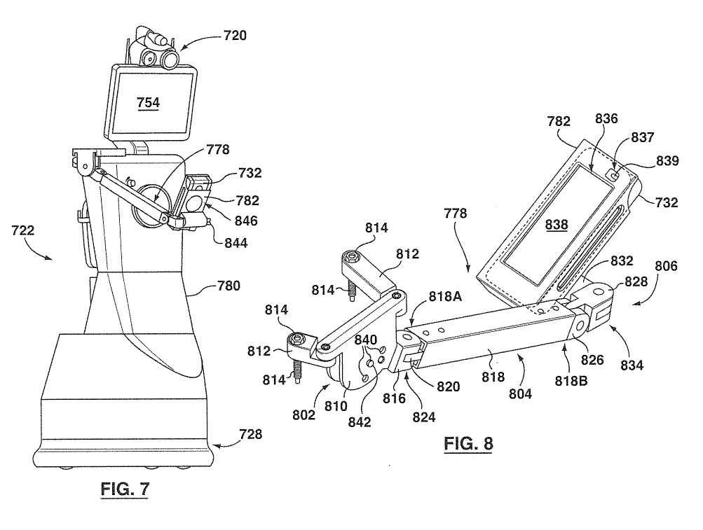

Reference is now made to FIG. 7, which shows an exemplary physical embodiment of a mobile medical telepresence robot 722 having an exemplary manually positionable arm 778, and which may be used as part of the exemplary medical telepresence system 610 shown in FIG. 6. As shown in FIG. 7, the mobile telepresence robot 722 is an RP-7® medical telepresence robot, offered by InTouch Technologies, Inc., doing business as InTouch Health Corporation and having an address at 90 Castilian Drive, Suite 200, Santa Barbara, Calif. 93117. Either the basic RP-7 model, or the RP-7i® model, which includes additional features, such as enhanced docking and enhanced audio, may be used. “RP-7” and “RP-7i” are registered trademarks of InTouch Technologies, Inc. in the United States. The RP-7 and RP-7i medical telepresence robots are merely examples of medical telepresence robots that may be used, and other medical telepresence robots, whether offered by InTouch Technologies, Inc. or by another provider, may also be used.

As can be seen in FIG. 7, the medical telepresence robot 722 includes robot body 780, a robot video camera 720, a locomotion system 728 and a robot video display 754. The medical telepresence robot 722 also includes a robot control system and a communication unit; these are not visible in FIG. 7 as they are contained inside the robot body 780. As will be described in greater detail below, in the embodiment shown in FIG. 7 a manually positionable arm 778 having a receptacle 782 is secured to the body 780 of the medical telepresence robot 722 to hold a medical treatment device 732. The manually positionable arm 778 is not part of the medical telepresence robot 722 as offered by InTouch Technologies, Inc.

In the exemplary physical embodiment shown in FIG. 7, the medical treatment device 732 is an N'Visione clinician programmer model 8840 offered by Medtronic Inc., having an address at 710 Medtronic Pkwy NE, LC-480, Minneapolis, Minn. 55432, U.S.A. The N'Vision clinician programmer model 8840 is a portable programming platform for implantable drug delivery and neurostimulation devices, such as deep brain stimulation devices, offered by Medtronic Inc., and includes a removable, wire-connected telemetry head for programming implanted devices and is provided with a touch screen for interactive programming. As such, because it can program an implanted treatment device in a patient, the N'Vision clinician programmer model 8840 enables actual treatment of a patient rather than merely monitoring and diagnosis. The N'Vision clinician programmer model 8840 is merely one example of a medical treatment device that may be used, and other medical treatment devices, whether offered by Medtronic Inc. or by another provider, may also be used.

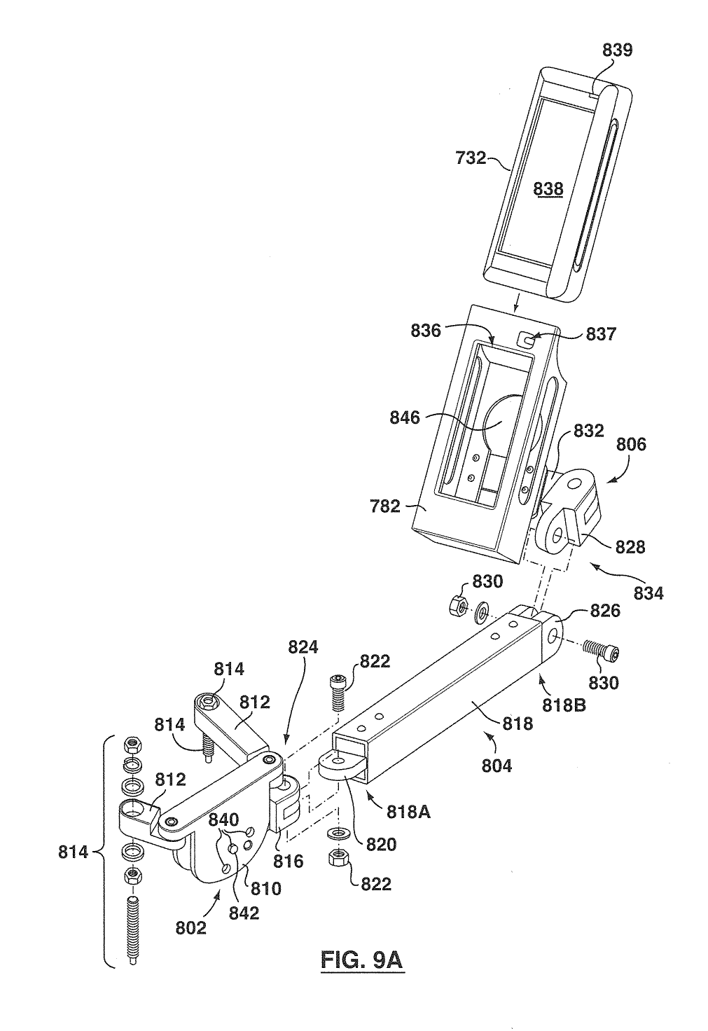

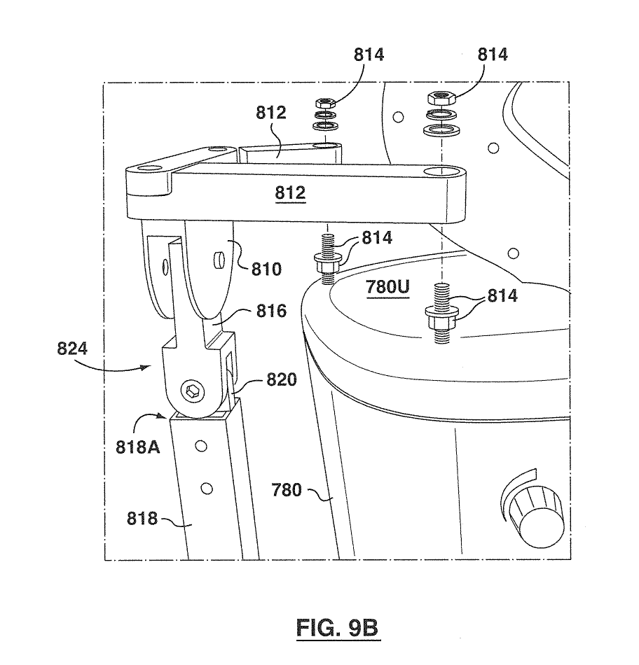

FIG. 8 shows a more detailed view of the exemplary manually positionable arm 778, and FIG. 9A shows an exploded view thereof. The arm 778 comprises three main portions, namely a shoulder portion 802 which is mounted to the medical telepresence robot 722, a main arm portion 804 extending from the shoulder portion 802, and a wrist portion 806 that carries the receptacle 782.

The shoulder portion 802 comprises a main scapular element 810 that is secured to the robot body 780 by way of its clavicle mounts 812, and a first humeral hinge half 816. As shown FIG. 9B, the main scapular element 810 is secured to the robot body 780 by removing the original equipment bolts from the upper portion 780U of the robot body 780 and then attaching the main scapular element 810 by way of two clavicle mounts 812, which are secured to the upper portion 780U of the robot body 780 by fastener assemblies 814 using the original holes from which the original equipment bolts were removed. Returning to FIGS. 8 and 9A, the main scapular element 810 pivotally receives the first humeral hinge half 816, to which the main arm portion 804 is mounted, as described below.

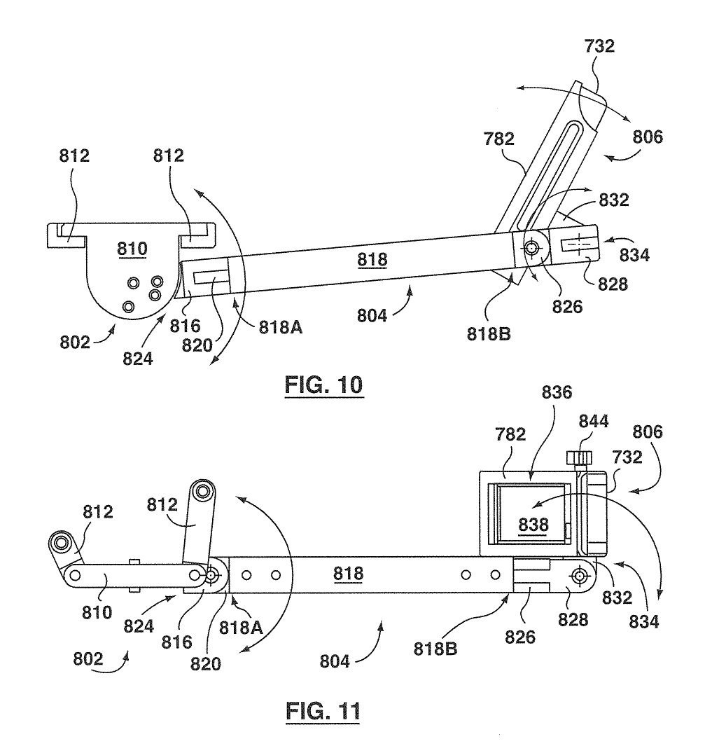

The main arm portion 804 comprises an elongate square tubular member 818 having a first end 818A and a second end 818B, as well as a second humeral hinge half 820 and a proximal wrist hinge segment 826. The second humeral hinge half 820 is mounted at the first end 818A of the elongate tubular member 818, and is pivotally mounted to the first humeral hinge half 816 by fasteners 822. The pivot axes of the first humeral hinge half 816 relative to the main scapular element 810, and of the second humeral hinge half 820 relative to the first humeral hinge half 816, are orthogonal to one another, so that the main scapular element 810, a first humeral hinge half 816 and second humeral hinge half 820 together form a humeral joint 824 which has two pivotal degrees of freedom, as shown in FIGS. 10 and 11.

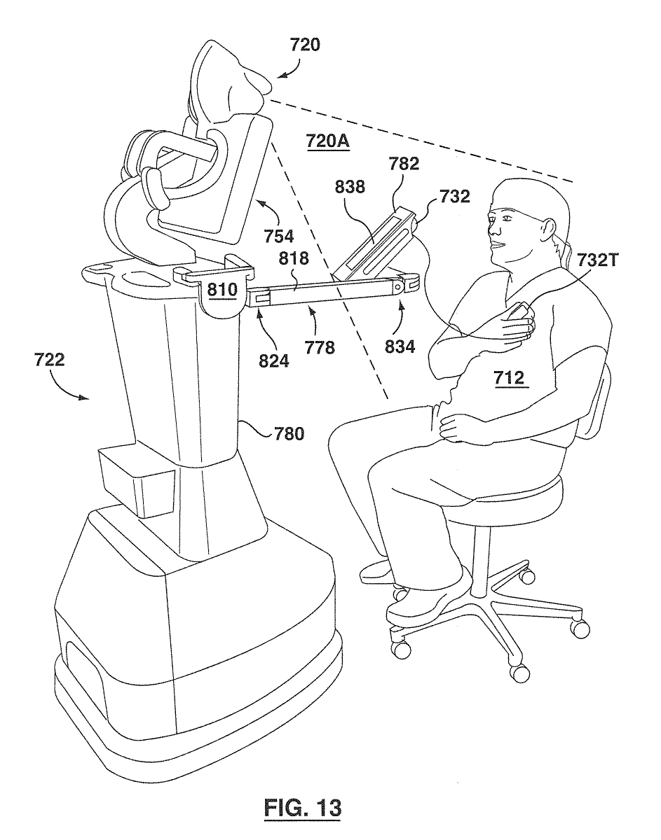

Returning to FIGS. 8 and 9A, the proximal wrist hinge segment 826 is mounted at the second end 818B of the elongate tubular member 818. The proximal wrist hinge segment 826 receives an intermediate wrist hinge segment 828, which is pivotally mounted to the first wrist hinge segment 826 by fasteners 830. The intermediate wrist hinge segment 828 in turn receives a distal wrist hinge segment 832, which is pivotally mounted to the intermediate wrist hinge segment 828. The pivot axes of the intermediate wrist hinge segment 828 relative to the proximal wrist hinge segment 826, and of the distal wrist hinge segment 832 relative to the intermediate wrist hinge segment 828, are orthogonal to one another. As such, the proximal wrist hinge segment 826, intermediate wrist hinge segment 828 and distal wrist hinge segment 832 together define a wrist joint 834 having two degrees of freedom, as shown in FIGS. 10 and 11. The distal wrist hinge segment 832 carries the receptacle 782, which includes a main aperture 836 for accessing the combined display and touch-screen interface 838 of the medical treatment device 732. The receptacle also includes a smaller aperture 837 to provide access to an emergency stop button 839 on the medical treatment device 732, and a circular aperture 846 opposite the main aperture 836, to provide access to the rewind spool of the medical treatment device 732 for retracting the telemetry head 732T (FIG. 13) thereof.

The first humeral hinge half 816 is mounted to the second humeral hinge half 820, and the proximal wrist hinge segment 826, intermediate wrist hinge segment 828 and distal wrist hinge segment 832 are mounted to one another, such that these parts will maintain their position relative to one another following manual adjustment; i.e. they maintain their position until moved with sufficient force, and maintain their new position when the force is removed. Construction of such an arrangement is within the capability of one skilled in the mechanical arts and is not described further. In addition, the receptacle 782 is pivotally mounted to the distal wrist hinge segment 832, and a screw-wheel is provided which when loosened permits pivotal adjustment of the receptacle 782 relative to the distal wrist hinge segment 832 and when tightened will lock the receptacle in position relative to the distal wrist hinge segment 832.

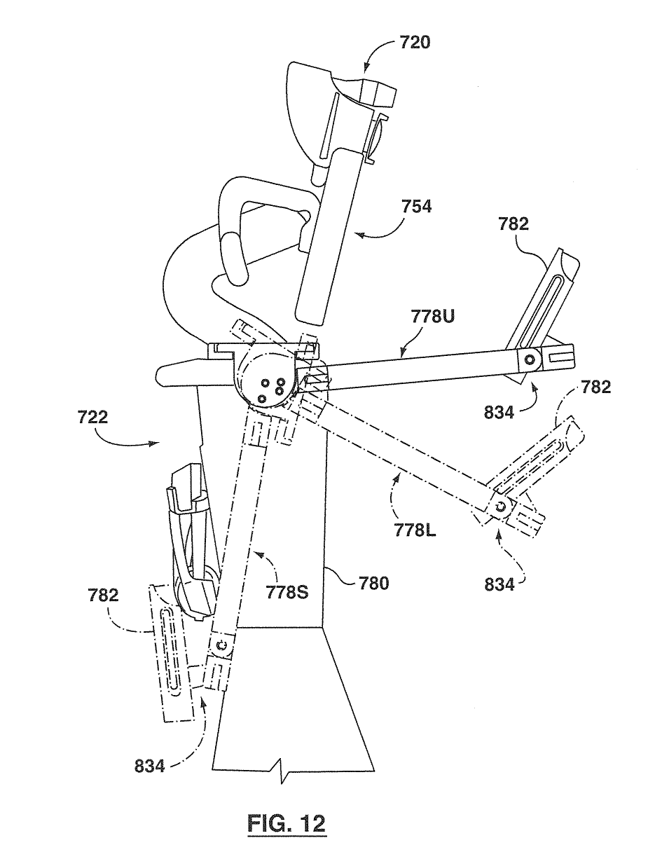

Continuing to refer to FIGS. 8 and 9A, the main scapular element 810 includes an arcuate arrangement of three locking apertures 840, into which can be received an outwardly biased detent 842 carried by the first humeral hinge half 816. The outwardly biased detent 842 can be selectively engaged with one of the three locking apertures 840 to maintain the first humeral hinge half 816, and hence the arm 778, in a selected angular orientation relative to the main scapular element 810. The outwardly biased detent 842 thus serves as a locking button. In particular, a user will push the outwardly biased detent 842 inwardly to disengage it from its respective locking aperture 840, and then move the aim 778 to the desired position relative to the main scapular element 810. When the outwardly biased detent 842 moves into registration with the locking aperture 840 associated with the desired position and the user releases the inward pressure, the outwardly biased detent 842 will move outwardly to engage with the locking aperture 840, and hence lock the first humeral hinge half 816 relative to the main scapular element 810. As shown in FIG. 12, this arrangement allows for the arm 778 to be locked in one of three primary positions, namely an upper operational position 778U for a standing patient or a tall seated patient, a lower operational position 778L for a seated patient of average height, and a storage position 778S, generally in line with the body 780 of the robot 722, for when the robot 722 is navigating about its environment or is being stored. In the storage position 778S, the wrist joint 834 is preferably angled so that the receptacle 782 extends inwardly, toward the body 780 of the robot 722, rather than outwardly therefrom.

Movement of the arm 778 into the desired position will generally take place under the guidance of the remotely located medical professional (e.g. the medical professional 616).

FIG. 13 shows the exemplary robot 722 with exemplary arm 778 holding exemplary medical treatment device 732 in use with a patient 712. Where the mobile telepresence robot is one of an RP-7 and RP-7i medical telepresence robot offered by InTouch Technologies, Inc. and the medical treatment device 732 is an N'Vision clinician programmer model 8840 offered by Medtronic Inc., the patient 712 should be seated approximately 12 to 30 inches away from the patient. As can be seen in FIG. 13, both the patient 712 and the medical treatment device 732, including the combined display and touch-screen interface 838 thereof, are within the field of view 720A of the robot video camera 720, and causing the patient 712 to be within the field of view of the robot video camera 720 also causes the robot video display 754 to face the patient 712.

The arm 778 is merely one exemplary embodiment of a manually positionable arm which can be used; a wide variety of other types of manually positionable arm may also be used.

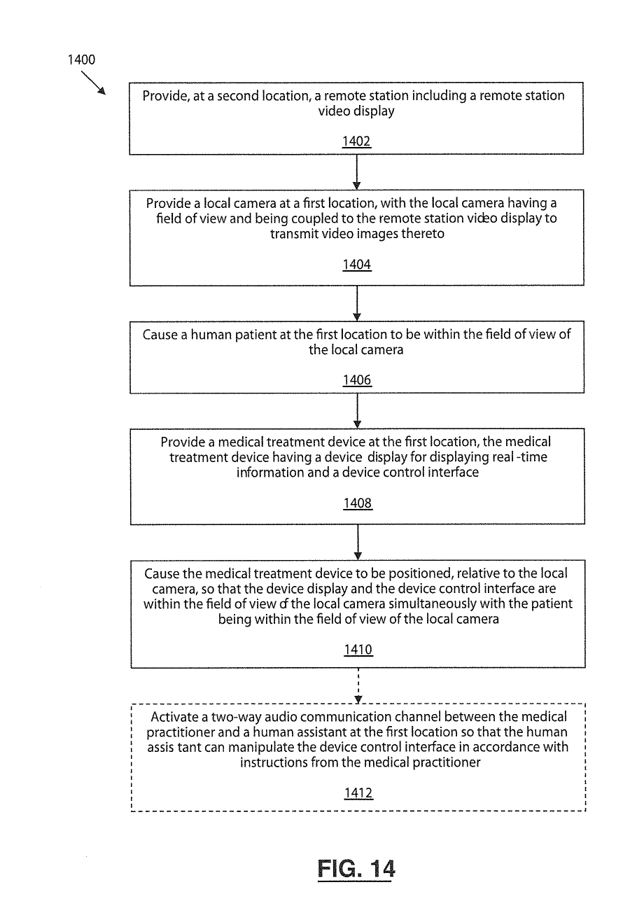

Referring now to FIG. 14, a medical telepresence system as described herein supports a method 1400 for enabling a medical practitioner to be telepresent at a first location when the medical practitioner is physically present at a second location that is physically remote from the first location. The method comprises a step 1402 of providing, at the second location, a remote station including a remote station video display and a step 1404 of providing a local video camera at the first location, with the local video camera having a field of view and being coupled to the remote station video display to transmit video images thereto. The steps 1402 and 1404 may be performed in any order. Step 1406 of the method comprises causing a human patient at the first location to be within the field of view of the local video camera. This can be achieved by bringing the local video camera to the patient, or by bringing the patient to the local video camera (e.g. if the local video camera is fixed) or moving both the local video camera and the patient toward one another. At step 1408 of the method, a medical treatment device is provided at the first location. The medical treatment device provided at step 1408 has a device display for displaying real-time information and a device control interface. Step 1410 of the method 1400 comprises causing the medical treatment device to be positioned, relative to the local video camera, so that the device display and the device control interface are within the field of view of the local video camera simultaneously with the patient being within the field of view of the local video camera. The method 1400 may comprise a further step 1412 of activating a two-way audio communication channel between the medical practitioner and a human assistant at the first location so that the human assistant can manipulate the device control interface in accordance with instructions from the medical practitioner, as shown schematically in FIGS. 1, 4 and 6, or merely so that the medical practitioner can communicate verbally with the patient.

As shown schematically in FIGS. 1 to 6, in a preferred embodiment of the method 1400 the step 1404 comprises providing a mobile robot 22, 222, 322, 422, 522, 622 at the first location, with the local video camera being a robot video camera 20, 220, 320, 420, 520, 620 carried by the mobile robot 22, 222, 322, 422, 522, 622, the step 1406 comprises positioning the mobile robot 22, 222, 322, 422, 522, 622 so that the patient 12, 212, 312, 412, 512, 612 is within the field of view 20A, 220A, 320A, 420A, 520A, 620A of the robot video camera 20, 220, 320, 420, 520, 620, and step 1410 comprises positioning the medical treatment device 32, 232, 332, 432, 532, 632 relative to the robot video camera 20, 220, 320, 420, 520, 620 so that the device display 34, 234, 334, 434, 534, 634 and the device control interface 36, 236, 336, 436, 536 are within the field of view 20A, 220A, 320A, 420A, 520A, 620A of the robot video camera 20, 220, 320, 420, 520, 620.

As also illustrated schematically in FIGS. 1 to 6, in a preferred embodiment the method 1400 further comprises providing a local video display, in the form of robot video display 54, 254, 354, 454, 554, 654, at the first location, with the remote station 40, 240, 340, 440, 540, 640 including a remote station video camera 56, 256, 356, 456, 556, 656 coupled, via the data processing system 42, 242, 342, 442, 542, 642, communications link 30, 230, 330, 430, 530, 630, communication unit 26, 226, 326, 426, 526, 626 and robot control system 24, 224, 324, 424, 524, 624, to the robot video display 54, 254, 354, 454, 554, 654 to transmit video images thereto. In this embodiment, the robot video camera 20, 220, 320, 420, 520, 620 and the robot video display 54, 254, 354, 454, 554, 654 are arranged relative to one another so that positioning the mobile robot 22, 222, 322, 422, 522, 622 so that the patient 12, 212, 312, 412, 512, 612 is within the field of view 20A, 220A, 320A, 420A, 520A, 620A of the robot video camera 20, 220, 320, 420, 520, 620 also positions the robot video display 54, 254, 354, 454, 554, 654 to face the patient 12, 212, 312, 412, 512, 612.

As shown schematically in FIGS. 2, 3, and 5, in a particular embodiment of the method 1400, the mobile robot 222, 322, 522 includes a first physical manipulator 270, 370, 570 and the device control interface 236, 336, 536 is positioned within a range of motion of the first physical manipulator 270, 370, 570. In this embodiment of the method 1400, the remote station 240, 340, 540 includes a remote station control interface 246, 346, 546 coupled, via the data processing system 242, 342, 542, communications link 230, 330, 530, communication unit 226, 326, 526 and robot control system 224, 324, 524, to the first physical manipulator 270, 370, 570 to remotely control the first physical manipulator 270, 370, 570. This enables the medical professional 216, 316, 516 to use the first physical manipulator 270, 370, 570 to manipulate the device control interface 236, 336, 536. In a more specific embodiment of the method 1400, as shown schematically in FIG. 2 in particular, the mobile robot 222 includes a second physical manipulator 272, which grasps the medical treatment device 232. In this embodiment, the remote station control interface 246 is also coupled, via the data processing system 242, communications link 230, communication unit 226 and robot control system 224, to the second physical manipulator 272 to remotely control the second physical manipulator 270.

In an alternate embodiment of the method 1400, as shown schematically in FIGS. 5 and 6, step 1410, that is, positioning the medical treatment device 532, 632 relative to the robot video camera 520, 620 comprises positioning a manually positionable arm 578, 678 on the mobile robot 522, 622 so that a receptacle 582, 682 on the arm 578, 678 is within the field of view 520A, 620A of the robot video camera 520, 620, and securing the medical treatment device 532, 632 in the receptacle 582, 682. The medical treatment device 532, 632 may be secured in the receptacle 582, 682 either before or after the arm 578, 678 is positioned.

FIGS. 5 and 6 have shown schematically, and FIG. 7 illustrates by way of a particular exemplary physical embodiment, a mobile medical telepresence robot 522, 622, 722. The mobile telepresence robot 522, 622, 722 comprises a robot body 580, 680, 780, a robot control system 524, 624, a communication unit 526, 626 operable to receive and transmit external communications (the robot control system and communication unit are not shown in FIG. 7), a locomotion system 528, 628, 728, a robot video camera 520, 620, 720 having a field of view 520A, 620A, 720A and a manually positionable arm 578, 678, 778 secured to the body 580, 680, 780 of the robot 522, 622, 722. The arm 578, 678, 778 has a receptacle 582, 628, 782 for securely receiving a medical treatment device 532, 632, 732, and is positionable so that the receptacle 582, 682, 782 is within the field of view 520A, 620A, 720A of the robot video camera 520, 620, 720 while a patient 512, 612 is also within the field of view 520A, 620A, 720A of the robot video camera 520, 620, 720. Alternatively, the arm 578, 678, 778 may have a fixed position and the robot video camera 520, 620, 720 may be adjustable so that the receptacle 582, 628, 782 is within the field of view 520A, 620A, 720A while the patient 512, 612, 712 is also within the field of view 520A, 620A, 720A. As shown in FIGS. 5, 6 and 7 the medical treatment device 532, 632, 732 has a device display 534, 634, for displaying real-time information and a device control interface 536, 636. Where, as shown in FIG. 7, the medical treatment device 732 is an N'Vision clinician programmer model 8840, the device display and device control interface are integrated into a single, touch-sensitive screen 838. The medical treatment device 532, 632, 732 is securely received in the receptacle 582, 628, 782 and the arm 578, 678, 778 and the robot video camera 520, 620, 720 are positioned relative to one another so that the device display 534, 634, 734 and the device control interface 534, 634, 734 are within the field of view 520A, 620A, 720A of the robot video camera 520, 620, 720.

The robot control system 524, 624 is coupled to the communication unit 526, 626, the locomotion system 528, 628, 728 and the robot video camera 520, 620, 720; the robot video camera 520, 620, 720 is thereby coupled to the communication unit 526, 626 and can transmit video images via the communication unit 526, 626 to a physically remote location. The mobile telepresence robot 522, 622, 722 further comprises a robot video display 554, 654, 754 coupled to the robot control system 524, 624 and thereby coupled to the communication unit 526, 626 for displaying images received from a remote video camera, such as the remote station video camera 556, 656.

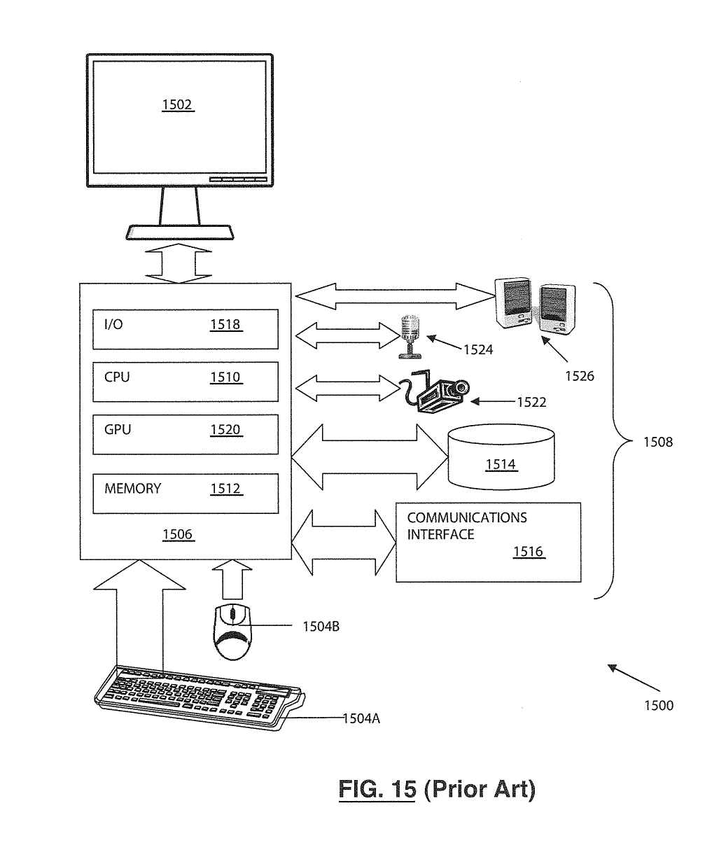

Medical telepresence systems, including mobile medical telepresence robots, may include or be implemented in association with any suitable computer or microprocessor-based system. An illustrative computer system in respect of which aspects of the medical telepresence systems, such as the remote station 40, 240, 340, 440, 540,640, may be implemented, is presented as a block diagram in FIG. 15. The illustrative computer system is denoted generally by reference numeral 1500 and includes a display 1502, input devices in the form of keyboard 1504A and pointing device 1504B, computer 1506 and external devices 1508, which may include a video camera 1522, microphone 1524 and speaker 1526. While pointing device 1504B is depicted as a mouse, it will be appreciated that other types of pointing device may also be used.

The computer 1506 may contain one or more processors or microprocessors, such as a central processing unit (CPU) 1510. The CPU 1510 performs arithmetic calculations and control functions to execute software stored in an internal memory 1512, preferably random access memory (RAM) and/or read only memory (ROM), and possibly additional memory 1514. The additional memory 1514 may include, for example, mass memory storage, hard disk drives, optical disk drives (including CD and DVD drives), magnetic disk drives, magnetic tape drives (including LTO, DLT, DAT and DCC), flash drives, program cartridges and cartridge interfaces such as those found in video game devices, removable memory chips such as EPROM or PROM, emerging storage media, such as holographic storage, or similar storage media as known in the art. This additional memory 1514 may be physically internal to the computer 1506, or external as shown in FIG. 15.

The computer system 1500 may also include other similar means for allowing computer programs or other instructions to be loaded. Such means can include, for example, a communications interface 1516 which allows software and data to be transferred between the computer system 1500 and external systems and networks. Examples of communications interface 1516 can include a modem, a network interface such as an Ethernet card, a wireless communication interface, or a serial or parallel communications port. Software and data transferred via communications interface 1516 are in the form of signals which can be electronic, acoustic, electromagnetic, optical or other signals capable of being received by communications interface 1516. Multiple interfaces, of course, can be provided on a single computer system 1500.

Input and output to and from the computer 1506 is administered by the input/output (I/O) interface 1518. This I/O interface 1518 administers control of the display 1502, keyboard 1504, external devices 1508 and other such components of the computer system 1500. The computer 1506 also includes a graphical processing unit (GPU) 1520. The latter may also be used for computational purposes as an adjunct to, or instead of, the (CPU) 1510, for mathematical calculations.

Aspects of the medical telepresence systems described herein, including mobile medical telepresence robots, may be implemented entirely in hardware or by way of a combination of hardware and software, including firmware, resident software, microcode, and the like, and which may be embodied in a computer program product accessible from a computer usable or computer readable medium providing program code for use by or in connection with a computer or any instruction execution system.

Although reference has been made herein to medical treatment devices, the present disclosure is not directed to methods of medical treatment, which remain entirely within the highly trained discretion of the relevant medical professional, but rather to creating circumstances that enable the medical professional to carry out medical treatment while physically remote from a patient.

The above description is intended in an illustrative rather than a restrictive sense. Variations to the exact embodiments described may be apparent to those skilled in the relevant art without departing from the spirit and scope of the claims set out below. It is intended that any such variations be deemed within the scope of this patent.

What is claimed is:

1. A method for enabling a medical practitioner to be telepresent at a first location when the medical practitioner is physically present at a second location physically remote from the first location, the method comprising:
providing, at the second location, a remote station including a remote station video display;
providing a local video camera at the first location, the local video camera having a field of view, the local video camera coupled to the remote station video display to transmit video images thereto;
causing a patient at the first location to be within the field of view of the local video camera;
providing a medical treatment device at the first location, the medical treatment device having a device display for displaying real-time information and a device control interface; and
causing the medical treatment device to be positioned, relative to the local video camera, so that the device display and the device control interface are within the field of view of the local video camera simultaneously with the patient being within the field of view of the local video camera.

2. The method of claim 1, wherein:
the step of providing a local video camera at the first location comprises providing a mobile robot at the first location, wherein the local video camera is a robot video camera carried by the robot;
the step of causing the patient to be within the field of view of the local video camera comprises positioning the mobile robot so that the patient is within the field of view of the robot video camera; and
the step of causing the medical treatment device to be positioned, relative to the local video camera, so that the device display and the device control interface are within the field of view of the local video camera, comprises positioning the medical treatment device relative to the robot video camera.

3. The method of claim 2, further comprising:
activating a two-way audio communication channel between the medical practitioner and a human assistant at the first location so that the human assistant can manipulate the device control interface in accordance with instructions from the medical practitioner.

4. The method of claim 2, wherein:
the robot includes a first physical manipulator;
the remote station includes a remote station control interface coupled to the first physical manipulator to remotely control the first physical manipulator; and
the device control interface is positioned within a range of motion of the first physical manipulator so that the first physical manipulator can manipulate the device control interface.

5. The method of claim 4, wherein:
the robot includes a second physical manipulator;
the remote station control interface is coupled to the second physical manipulator to remotely control the second physical manipulator; and
wherein the medical treatment device is grasped by the second physical manipulator.

6. The method of claim 2, wherein a manually positionable arm is secured to the robot, and wherein the step of positioning the medical treatment device relative to the robot video camera comprises:
positioning the manually positionable arm so that a receptacle on the arm is within the field of view of the robot video camera; and
securing the medical treatment device in the receptacle;
wherein the steps of positioning the manually positionable arm and securing the medical treatment device in the receptacle are carried out in any order.

7. The method of claim 1, further comprising:
providing a local video display at the first location;
wherein the remote station includes a remote station video camera coupled to the local video display to transmit video images thereto; and
wherein the local video camera and the local video display are arranged relative to one another so that causing the patient at the first location to be within the field of view of the local video camera also causes the local video display to face the patient.

8. The method of claim 2, wherein:
the robot includes a robot video display;
the remote station includes a remote station video camera coupled to the robot video display to transmit video images thereto; and
wherein the robot video camera and the robot video display are arranged relative to one another so that positioning the mobile robot so that a patient is within the field of view of the robot video camera also positions the robot video display to face the patient.

9. A medical telepresence system, comprising:
at a first location, a local video camera having a field of view;
the local video camera being coupled to a communications link for transmitting video images to a second location physically remote from the first location; and
at the first location, a medical treatment device having a device display for displaying real-time information and a device control interface;
characterized in that the medical treatment device is positioned so that the device display and the device control interface are within the field of view of the local video camera and the field of view also simultaneously accommodates a patient.

10. The medical telepresence system of claim 9, further comprising a two-way audio communication channel between the first location and the second location.

11. The medical telepresence system of claim 9, wherein the local video camera is a robot video camera carried by a mobile telepresence robot.

12. The medical telepresence system of claim 11, wherein:
the robot includes a robot control system coupled to the communications link;
the robot video camera is coupled to the robot control system and thereby coupled to the communications link;
the robot includes a locomotion system coupled to the robot control system;
further comprising a remote station at the second location, the remote station coupled to the communications link and thereby coupled to the robot control system to control the robot.

13. The medical telepresence system of claim 12, wherein:
the robot includes a first physical manipulator coupled to the robot control system;
the remote station includes a remote station control interface coupled to the first physical manipulator through the communications link and the robot control system to remotely control the first physical manipulator; and
the device control interface is positioned within a range of motion of the first physical manipulator so that the first physical manipulator can manipulate the device control interface.

14. The medical telepresence system of claim 13, wherein:
the robot includes a second physical manipulator coupled to the robot control system;
the remote station control interface is coupled to the second physical manipulator through the communications link and the robot control system to remotely control the second physical manipulator; and
wherein the medical treatment device is grasped by the second physical manipulator.

15. The medical telepresence system of claim 11, wherein:
a manually positionable arm is secured to a body of the robot;
the arm has a receptacle for securely receiving the medical treatment device;
the medical treatment device is securely received in the receptacle; and
the arm is positioned so that the receptacle is within the field of view of the robot video camera.

16. The medical telepresence system of claim 9, further comprising:
a local video display at the first location;
a remote station including a remote station video camera coupled to the local video display to transmit video images thereto; and
wherein the local video camera and the local video display are arranged relative to one another so that causing the patient at the first location to be within the field of view of the local video camera also causes the local video display to face the patient.

17. The medical telepresence system of claim 12, wherein:
the robot includes a robot video display;
the remote station includes a remote station video camera coupled to the robot video display to transmit video images thereto; and
wherein the robot video camera and the robot video display are arranged relative to one another so that positioning the mobile robot so that the patient is within the field of view of the robot video camera also positions the robot video display to face the patient.

18. A mobile medical telepresence robot, comprising:
a robot video camera having a field of view;
the robot video camera being coupled to a communication unit for transmitting video images to a physically remote location; and
a manually positionable arm secured to a body of the robot;
the arm having a receptacle for securely receiving a medical treatment device;
at least one of the arm and the robot video camera being positionable so that the receptacle is within the field of view of the robot video camera while a patient is also within the field of view of the robot video camera.

19. The mobile medical telepresence robot of claim 18, wherein a medical treatment device having a device display for displaying real-time information and a device control interface is securely received in the receptacle and the arm and the robot video camera are positioned relative to one another so that the device display and the device control interface are within the field of view of the robot video camera.

20. The mobile medical telepresence robot of claim 19, wherein the mobile telepresence robot comprises:
the robot body;
the robot video camera;
the communication unit,
a robot control system coupled to the communication unit;
a locomotion system coupled to the robot control system;
wherein the robot video camera is coupled to the robot control system and thereby coupled to the communication unit; and
wherein the communication unit is operable to receive and transmit external communications.

21. The mobile medical telepresence robot of claim 20, further comprising a robot video display coupled to the robot control system and thereby coupled to the communication unit to display images from a remote video camera.
